(19) United States
(12) Patent Application Publication
(10) Pub. No.: US 2004/0146539 A1
(43) Pub. Date: Jul. 29, 2004

(54) Topical Nutraceutical Compositions with Selective Body Slimming and Tone Firming Antiaging Benefits

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/248,508

(22) Filed: Jan. 24, 2003

(51) Int. Cl. A61K 7/00

(52) U.S. Cl. 424/401

(57) ABSTRACT

I have discovered cosmetic or topical pharmaceutical compositions for external body part or organ slimming, firming, cellulite reduction, fat-reduction, and obesity control benefits that are in synergistic combination with benefits for the treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, spider veins reduction, blood microcirculation (venous insufficiency) improvement, UVA/UVB protection of skin, and skin redness reduction. These compositions thus provide multiple combinations of skin and external body part or organ enhancement benefits that can be selective and specific for external body parts and organs such as face, chin, cheeks, arms, “love handles” in abdomen area, eye lids and eye zone, neck, breasts, thighs, and hips. These compositions include a body beneficial composition selected from certain nutraceutical, cosmetic, and pharmaceutical ingredients, a composition to promote collagen and elastin synthesis in the skin, and a cosmetically or pharmaceutically acceptable delivery system.

### BACKGROUND OF INVENTION

The enhancement of physical appearance occupies greater focus in human life than nearly all other daily life-related concerns combined. There are far more consumer products available for the beautification of human body than for the treatment of human ailments. The improvement of body tone and appearance is a growing, multibillion-dollar industry encompassing cosmetic, nutraceutical, pharmaceutical, and physical therapy disciplines. The consumer attention is focused on newest miracle ingredient in age-defying, anti-wrinkle, body firming, fat burning, varicose reducing, and lean body mass increasing concoctions, promotions, witchcraft, and claims.

A number of nutraceutical ingredients have found applications for body weight management, body firming, body slimming, skin tightening, reduction of varicose veins, and reduction of excess fatty tissue. Similarly, several cosmetic ingredients have been found useful for the improvement of skin appearance via their skin beneficial functions that include skin resurfacing, skin wrinkles reduction, skin pigmentation reduction, skin whitening, and antiaging effects. While cosmetic ingredients are usually applied directly on the skin, the nutraceutical ingredients are most commonly ingested for their claimed benefits. It would thus be advantageous if nutraceutical ingredients that have body slimming and other such fat reducing properties can be developed in topical compositions. As can be appreciated by most consumers, the ingestion of nutraceutical ingredients usually has an affect on the overall body that usually is not controllable or specific for selective external body organs. The topical application of such nutraceutical ingredients can alleviate this problem; as such compositions can be applied directly on the external organ or body part where a desired benefit is sought. For example, such compositions can be applied directly on chin to cure “double chin” problems, or applied on the abdomen to reduce fatty “love handles”.

Moreover, the combination of such benefits with cosmetic benefits of antiaging, skin whitening, skin wrinkle reduction, varicose veins reduction, etc. can provide consumer benefits that can be targeted selectively for external body organs of choice. Another example is MSM (methylsulfonylmethane), a very popular nutraceutical ingredient for arthritis pain. The ingestion of this ingredient is known to cause bad breath and poor body smell due to the distribution of such odor causing metabolites of this ingredient throughout the body via bloodstream. A topical nutraceutical product using this ingredient can circumvent that unpleasant consumer problem, and also offer direct application on the body organ where arthritis pain relief is desired. By such topical applications the amount that is absorbed into bloodstream and carried throughout rest of the body is significantly reduced or even eliminated.

Since it is very difficult to formulate ingestible ingredients for topical applications, as the composition of the delivery systems for such applications to provide optimal skin penetration, bioavailability, synergism, and product stability become important. Moreover, many such nutraceutical ingredients have properties, such as being sticky or greasy, that can pose problems in their cosmetic acceptability in topical compositions especially when such ingredients are combined with cosmetic ingredients for synergistic combination benefits.

It would thus be highly desirable to develop topical compositions of nutraceutical ingredients to provide external body part or organ selective topical benefits. It would be additionally desirable if such topical nutraceutical compositions can be combined with certain complementary or synergistic cosmetic ingredients to provide topical compositions with dual benefit of both nutraceutical and cosmetic skin and body organ beneficial ingredients. Currently most nutraceutical compositions for ingestion mode of administration are mixtures of several ingredients that are randomly selected to offer multiple benefits. It would thus be advantageous to review the mode of biochemical mechanisms by which each of such nutraceutical ingredients function, as that would permit a more scientific combination of suitable synergistic compositions that may avoid undesirable inter-drug or drug-cosmetic ingredients biochemical interactions and chemical reactivity. It would also be beneficial to review prior art knowledge in this field.

Topical compositions for body slimming using certain plant or nutraceutical ingredients have been disclosed in prior art, for example in U.S. patent application Ser. No. 20,030,007,988 (to O. Courtin). However, this disclosure comprises a specific plant extract containing a plant natriuretic peptide. These compositions were tested by oral administration route, and their benefits, if any, by topical application in a scientifically designed formulation have not been disclosed.

U.S. patent application Ser. No. 20,030,003,063 (to W. K. Kim et al.), a UV blocking and slimming composition is disclosed. Again, this disclosure is based solely on one slimming ingredient from Laminaria extract. It is thus specific to this one ingredient and does not teach any synergistic benefits that may be derived when combinations of several biochemically compatible slimming or body fat reducing ingredients are used.

U.S. Pat. No. 5,804,596 (to Majeed et al.), a method of promoting lean body mass in an individual is disclosed, comprising administering orally to the individual a lean body mass promoting effective amount of forskohlin extract from Coleus Forskohlii plant. Neither any topically effective compositions, nor any combinations of forskohlin extract with skin beneficial antiaging or anti-wrinkle ingredients have been disclosed.

U.S. patent application Ser. No. 20,020,187,943 (to Majeed et al.), the combination of hydroxycitric acid, obtained from Garcinia cambogia plant, and antioxidants, garcinol and anthocyanins, was claimed to give synergistic therapeutic effects in murine and human studies that includes a reduction in total body weight and body mass index, a reduction in body fat, an increase in lean body mass and content of body water, and a reduction in perceived appetite level. Again, these compositions were tested by oral administration route, and their benefits, if any, by topical application are not disclosed.

Another nutraceutical ingredient, L-carnitine, has found applications for the facilitation of the metabolism of lipids by oral administration route, as disclosed in U.S. patent application Ser. No. 20,020,111,383 (to K. Hassen).

U.S. patent application Ser. No. 20,020,137,691 (To H. Murad) claims chromium picolinate to provide fat burning efficacy in combination with other synergistic ingredients, which are principally oral pharmaceutical compositions. Although Murad does disclose topical compositions of chromium picolinate in combination with other skin beneficial ingredients, such topical compositions also include sugar derivatives, some of which are not suitable for cosmetically elegant compositions due to their stickiness on skin surface from such topical applications.

U.S. Pat. No. 6,340,669 (to Cestaro et al.,) discloses certain Lipoprotein complexes comprising a lipase-inhibiting protein component, and/or amylase-inhibiting protein component and a phospholipid component, compositions containing said complexes in combination with a polysaccharide component, pharmaceutical formulations and foods containing said complexes or compositions, processes for the preparation of said complexes and compositions, which are capable of reducing the weight increase following hypercaloric diets and of exerting hypocholesterolemizing, hypotriglyceridemizing and antioxidizing activities. However, such compositions are suitable for oral administration only, and their topical application to provide any body part slimming and fat-burning benefits have not been claimed.

U.S. patent application Ser. No. 20,030,004,215 (to Van Laere et al.) discloses a carbohydrate absorption inhibitor selected from the group consisting of polyphenols, gymnemic acid, phaseolamin, roselle tea, lotus, arabinose, inosine, adenosine, evening primrose extract, banaba extract, indigestible dextrin, and mixtures thereof in combination with an alpha.-hydroxy carboxylic acid component and a pharmaceutically acceptable excipient. The formulation of these ingredients in any topically effective composition for combination benefits has not been disclosed.

U.S. patent application Ser. No. 20,020,197,301 (to M. W. Pariza) discloses a method for controlling body fat in a human or nonhuman animal includes the step of reducing lipoxygenase activity in an animal. Lipoxygenase activity can be reduced by reducing the enzyme activity or by lowering the enzyme level. Reduced lipoxygenase activity correlates with reduced cell-associated LPL activity and with reduced cellular triacylglyceride level. The lipoxygenase inhibitor is selected from NDGA, AA861, Indomethacin, ETYA, SHA, Baicalein, 3,4-dihydroxycinnamic acid, cinnamyl-3,4-dihydroxy-a-cyanocinnamate, Gossypol, 5,6-dehydro arachidonic acid, Baeomycesic acid, Baicalein monohydrate, 3,4-dihydroxyphenyl ethanol, 4,5-dehydro docosahexaenoic acid, eicosatriynoic acid, 5-HETE lactone, 5(S)-HpETE, 12(S)-HpETE, 15(S)-HpETE, 15(S)-HETrE, 9,12-octadecadiynoic acid, a-pentyl-3-(2-quinolinylmethoxy)-benzenemethanol, BHA, BHT, 3-amino-1-/m-(trifluoromethyl) phenyl/-2-pyrazoline, and 6,9-diepoxy-6,9-phenylimino-delta 6,8-prostaglandin I. Any topically applied compositions of this ingredient for any body slimming, body fat reduction, or cellulite reduction benefits have not been disclosed.

U.S. Pat. No. 5,165,935 (to Andre et al.) discloses a topical cosmetic composition based on kola seed extract for cellulite reduction. However, the use of a single ingredient in such topical cosmetics makes such products of limited utility and benefit. It would be advantageous to include other synergistic body slimming or antiaging ingredients with kola extract, for example, for significantly improved benefits.

U.S. Pat. No. 5,709,864 (to Andre et al.) discloses that the extract of the plant of genus Tephrosia is useful in preparing a cosmetic, pharmaceutical, and in particular dermatological composition, presenting body-slimming, pigmenting, anti-aging, or anti-inflammatory activity. Again, this disclosure is based on a single ingredient with multiple functions. It would thus be advantageous to develop synergistic mixtures of this ingredient with other body beneficial cosmetic compositions, which have not been disclosed in the prior art.

U.S. Pat. No. 5,658,576 (to Soudant) a body slimming composition which contains, as the sole slimming agent, an alpha-tocopherol, benzyl nicotinate, xanthinol or hexyl nicotinate, or alpha-tocopherol acetate. Some of these ingredients, such as benzyl nicotinate and hexyl nicotinate, are highly irritating to skin, hence not very useful.

U.S. Pat. No. 5,962,482 (to Bissett) relates to a method for combating cellulite or reducing localized fatty excesses which comprises administering to a person having cellulite or localized fatty excesses a body slimming amount of a composition containing niacinamide. These compositions can contain additional beneficial ingredients, selected from the group consisting of hydroxy acids, desquamatory agents, sunscreens, anti-oxidants, retinoids and mixtures thereof. These compositions, although they do contain mixtures of several beneficial ingredients, they do not combine such ingredients in any synergistic topical compositions to provide combination benefits from such cosmetic compositions.

U.S. Pat. No. 6,114,336 (to Blanc-Ferras et al.) relates to a cosmetic composition containing at least one NPY-antagonist comprising a neuropeptide Y antagonist and an .alpha.2 antagonist in combination with cosmetically acceptable excipient, wherein the neuropeptide Y antagonist is a fermentation product of the strain Streptomyces sp SEBR 2794 as well as productive mutants thereof. This disclosure is based on a single specific ingredient that is not commonly available, hence of limited utility to broader consumer markets.

U.S. patent application Ser. No. 20,020,136,785 (to C. S. Yuan) relates to the methods of screening for the active compound from berries from plants of the Panax genus that decreases blood glucose and decreases body weight. It is contemplated that the active compound may comprise a ginsenoside or a combination thereof. The benefits of topical application of this ingredient for the same desired attributes of body weight reduction or slimming was not reported.

U.S. patent application Ser. No. 20,020,187,204 (to B. Alviar et al.) discloses a diet composition for managing body weight including effective amounts of Garcinia cambogia extract, Gymnema sylvestre extract, chromium picolinate, vanadium compound, L-carnitine, and conjugated linoleic acid. The daily effective amounts are administered in three approximately equal doses in conjunction with the daily meals. The diet composition is also administered in conjunction with a restricted-calorie diet. The diet composition optionally includes effective amounts of kola nut extract, dehydrated parsley, and lemon bioflavonoids. Although this patent does disclose the application of synergistic combinations of several ingredients for body weight management, all are ingestible compositions useful for a dietary intake and not suitable for topical application.

U.S. Pat. No. 6,384,087 and U.S. patent application Ser. Nos. 20,020,132,014, 20,020,132,014, and 20,020,192,264 (all to M. B. Zemel et al.) provides methods of inducing the loss of adipose tissue by providing a diet high in calcium. In one aspect of the invention, the calcium is provided in the form of dairy products. In yet another aspect of the invention, calcium is provided in the form of a dietary supplement, such as calcium carbonate, of vitamin supplements. Methods of suppressing [Ca.sup.2+].sub.i levels in individuals are also provided. The subject invention also provides methods of stimulating lipolysis, inhibiting lipogenesis, and increasing the expression of white adipose tissue uncoupling protein 2 (UCP2). The subject invention also provides methods of increasing the core temperature of an individual. These compositions are all orally administered as diet compositions, and not suitable for topical applications.

U.S. patent application Ser. No. 20,020,058,075 (to D. Jones) claims materials derived from Citrus plants can be administered orally to humans for the purpose of producing or maintaining weight loss as well as for improving the person's physical performance and increasing the person's lean muscle mass. The Citrus materials include those portions of the plant that are normally considered waste or inedible, such as the leaves, peel, and immature, unripe fruit. The materials contain at least one of the alkaloids from the group consisting of synephrine, hordenine, octopamine, tyramine and N-methyltyramine. Two species, Citrus aurantium and Citrus reticulata, are particularly useful. The materials can be administered in their natural form or as extracts, and can be administered in various ways including capsules and tablets. The Citrus materials may also be used as a tea. For weight loss and weight control, the materials can be administered concurrently with caloric restriction or in the absence of caloric restriction. The materials may also be administered for the purpose of increasing muscle mass concurrently with a high protein diet as well as with an exercise program. These compositions are all orally administered as diet compositions, and not suitable for topical applications.

U.S. patent application Ser. No. 20,020,081,315 (to D. P. Katz et al.) discloses a composition for treating insulin-dependent diabetes, reducing body fat, improving insulin sensitivity, reducing hyperglycemia, and reducing hypercholesterolemia with at least one chromium complex and a conjugated fatty acid or conjugated fatty alcohol is disclosed. A method of treating a subject suffering from insulin-dependent diabetes by administering a composition that includes at least one chromium complex and a conjugated fatty acid or conjugated fatty alcohol is similarly provided. The administration of a composition containing an effective dose of at least one chromium complex and a conjugated fatty acid or conjugated fatty alcohol for the treatment of obesity is likewise provided. Although synergistic mixtures are claimed, these compositions are all orally administered as diet compositions, and not suitable for topical applications.

U.S. Pat. No. 6,433,015 (to Meyer) discloses a process for reducing the body weight of overweight humans and domestic animals, such as dogs and cats, by administering creatine is provided. Creatine is administered at a daily dose of 0.15 mg-15 mg per kilogram of body weight by various methods, including oral administration, injection, infusion, and suppositories for rectal application. Also provided is the use of creatine for manufacturing a medicament containing creatine that can be administered by the process of the invention. These compositions are all orally administered as diet compositions, and not suitable for topical applications.

U.S. Pat. No. 6,399,565 (to Asada et al.) and U.S. Pat. No. 6,335,319 (to Ng) disclose that administration of an authentic human growth hormone or a human growth hormone fragment to human adults can improve body composition, stimulate lipolysis, increase serum insulin-like growth factor I or be used in replacement therapy for human growth hormone-deficient patients. These compositions are all orally administered as diet compositions, and not suitable for topical applications.

U.S. Pat. No. 6,307,080 (to Pischel et al.) teaches that salts of pyruvic acid, called pyruvates, have valuable physiological, therapeutic and dietary properties. Pyruvates are used to increase stamina and strength in the sports sector, for weight reduction and body fat reduction. These compositions are all orally administered as diet compositions, and not suitable for topical applications.

U.S. Pat. No. 6,020,378 (to Cook et al.) discloses a method of reducing body fat in an animal without reducing weight gain or feed efficiency, said method comprising administering to said animal a safe and effective amount of a composition comprising 9-cis, 11-trans conjugated linoleic acid and 10-trans, 12-cis conjugated linoleic acid isomers, the composition comprising more 9-cis, 1-trans conjugated linoleic acid isomer than 10-trans, 12-cis conjugated linoleic acid isomer, wherein the isomers are administered orally to the animal in a food. These compositions are all orally administered as diet compositions, and not suitable for topical applications.

U.S. Pat. No. 5,760,047 (to Cincotta et al) disclose a method of weight and/or body-fat reduction comprising a (preferably moderate) reduction in the caloric intake of a subject in need of such treatment in combination with administration to said subject of a prolactin inhibitor. Additionally, this invention is directed to an improvement in a method for altering and/or resetting prolactin profiles (and thereby controlling one or more metabolic disorders such as obesity, excessive body fat, hyperlipidemia, hyperlipoproteinemia, hyperglycemia, hypercholesterolemia, hyperinsulinemia, insulin resistance, glucose intolerance, and Type II diabetes) comprising administration to a subject in need of such treatment of a prolactin inhibitor at a predetermined time or times during a 24-hour period in combination with a (preferably moderate) reduction of the caloric intake of said subject. These compositions are all orally administered as diet compositions, and not suitable for topical applications.

U.S. Pat. No. 6,395,296 and 6,160,172 (both to Balasubramanyam et al) relates to a new soluble double metal salt of group IA and IIA of (−) hydroxycitric acid, process of preparing the same and its use in beverages and other food products without effecting their flavor and properties. This product with >98% purity can be used safely not only as a food supplement in various nutraceutical formulations and beverages but also for effecting obesity control. These compositions are all orally administered as diet compositions, and not suitable for topical applications.

U.S. Pat. No. 5,756,467 (to Kagawa et al.) provides a peptide having an amino acid sequence of Val-Tyr-Pro or Val-Thr-Leu and having the ability to inhibit adipocyte differentiations, adipocyte differentiation inhibiting agent incorporating therein as an active component the peptide or a proteolysate characterized by containing not less than 0.1% by weight of the peptide, a specific health food, and a feed. This invention permits prevention and therapy of obesity. These compositions are all orally administered as diet compositions, and not suitable for topical applications.

U.S. Pat. No. 4,897,390 (to Ruhe) discloses that the most efficient weight reduction strategy would simultaneously decrease caloric intake while increasing caloric expenditure. To this end, dehydroepiandrosterone (“DHEA”) is a promising antiobesity agent. The exact mechanism by which DHEA affects weight loss is not known. However, experimental evidence suggests that DHEA inhibits particular metabolic enzymes (and thus caloric intake) while simultaneously increasing the overall metabolic rate (and therefore caloric expenditure). For instance, as to its inhibitory effect, DHEA is known to inhibit mammalian glucose-6-phosphate dehydrogenase. DHEA is also a known inhibitor of NADH oxidase, the first enzyme of the cytochrome system. In addition, DHEA is known to directly inhibit fatty acid synthetase. Finally, DHEA is known to inhibit 11.beta.-hydroxylase, an enzyme complex that converts 11-deoxycorticosterone to aldosterone and 11-deoxycortisol to cortisol. Since hypercorticism is a prominent feature of genetically obese mice, occurring after the onset of obesity, DHEA could limit the production of glucocorticoids and restore a more normal hormonal balance, thereby decreasing the severity of obesity. U.S. Pat. No. 4,680,289 (to Applezweig) discloses obesity control by DHEA metabolites, such as Etiocholanolone. The topical application of DHEA and its derivatives would thus offer advantages in combination with synergistic ingredients for selective slimming of body parts in combination with other desirable beautifying cosmetic effects, as none of such are yet known.

U.S. Pat. No. 6,506,420 and U.S. patent application Ser. No. 20,020,137,729 (both to J. G. Babish) describe a composition for minimizing the absorption of triglycerides and fats in the gastrointestinal tract. The composition comprises psyllium and chitosan in a ratio range of 2:1 to 11:1 by weight, preferably 2:1 to 9:1, and most preferably 2:1 to 4:1 to provide a synergistic fat-binding effect. These compositions are all orally administered as diet compositions, and not suitable for topical applications. Since both psyllium and chitosan can bind fat, their use in topical compositions to treat surface skin problems such as acne and rosacea, which are usually caused by excess fat on skin surface, could provide such desirable consumer benefits, which are yet undisclosed.

U.S. Pat. No. 6,323,189 (to L. Hardinge) describes stable chitosan-containing compositions (especially a stable liquid suspension composition) suitable for a weight treatment program. These compositions are all orally administered as diet compositions, and not suitable for topical applications.

U.S. Pat. No. 6,274,550 (to Prasad et al.) describes a proteoglycan (“Azaftig”) with a molecular weight of approximately 24,000 Dalton. Azaftig has been shown to bind to receptors on fat cell membranes, and to cause lipolysis. Azaftig does not bind to muscle cell membranes, or cause proteolysis in muscle tissue. Azaftig detection in urine or other body fluids will allow early identification of patients in which weight loss may become a problem. Azaftig may also aid fat loss in humans in which obesity is a threat to health. The topical application of Azaftig and its derivatives would thus offer advantages in combination with synergistic cosmetic ingredients for selective slimming of body parts in combination with other desirable beautifying cosmetic effects, as none of such are yet known.

U.S. Pat. No. 6,350,730 (to Friedman) claims nucleotide sequences corresponding to the murine and human OB gene, and two isoforms thereof, and proteins expressed by such nucleotides or degenerate variations thereof, that demonstrate the ability to participate in the control of mammalian body weight and that have been postulated to play a critical role in the regulation of body weight and adiposity. The topical application of such derivatives would thus offer advantages in combination with synergistic cosmetic ingredients for selective slimming of body parts in combination with other desirable beautifying cosmetic effects, as none of such are yet known.

U.S. patent application Ser. No. 20,020,112,253 (to S J. Wakil et al.) describes the role of acetyl-CoA carboxylase through its product malonyl-CoA in regulating fatty acid oxidation and synthesis, glucose metabolism and energy homeostasis. It discloses transgenic mice with inactivating mutations in the endogenous gene for the acetyl-CoA carboxylase 2 isoform of acetyl-CoA carboxylase. Inactivation of acetyl-CoA carboxylase 2 results in mice exhibiting a phenotype of reduced malonyl-CoA levels in skeletal muscle and heart, unrestricted fat oxidation, and reduced fat accumulation in the liver and fat storage cells. As a result, the mice consume more food but accumulate less fat and remain leaner than wild-type mice fed the same diet. These results demonstrate that inhibition of ACC2 acetyl-CoA carboxylase could be used to regulate fat oxidation and accumulation for purposes of weight control. The topical application of inhibitors of ACC2 acetyl-CoA carboxylase enzyme would thus offer advantages in combination with synergistic cosmetic ingredients for selective slimming of body parts in combination with other desirable beautifying cosmetic effects, as none of such are yet known.

U.S. patent application Ser. No. 20,010,041,708 (to Yuan-Di Chang Halvorsen, et al.) relates to a method for combating cellulite or reducing localized fatty excesses which comprises administering to a person having cellulite or localized fatty excesses a body slimming amount of a composition containing 10-trans, 12-cis conjugated linoleic acid. Although this patent does disclose both orally administered and topical cosmetic compositions, only a single ingredient (conjugate linoleic acid, CLA) is utilized in such compositions and any synergistic combinations are not utilized for enhanced efficacy or combination benefits.

U.S. Pat. No. 6,193,975 (to Bonte et al.) describes the use of Potentilla erecta extract for the improvement of the synthesis of collagen VII. The use of this ingredient as an antioxidant or body fat slimming has not been reported.

The development of topical compositions (especially those that can also combine the desirable benefits of cosmetic compositions) that utilizes body beneficial nutraceutical ingredients (a number of which have been described in various citations above) for body shape management and body tone enhancement has thus been very limited. Most nutraceutical compositions are administered orally. This is of significant disadvantage in many cases due to poor taste, poor absorption from digestive system, degradation of active principles after their absorption into bloodstream by body enzymes, and rapid metabolism by liver before any active ingredients present in such nutraceutical compositions can actually reach the site of affliction or body organ where their benefits are desired.

The sports nutrition and weight loss market in the U.S. totaled nearly 10 billion U.S. Dollars in 2001. Of various categories, weight loss segment of the nutraceuticals market posted a whopping 20% growth from 2000 to 2001. The usual weight loss or slimming compositions, which are taken orally, usually have an affect on all body parts, and they can not be made selective or customized to match the biochemistry of just one body part, or a group of body parts. The weight loss, slimming, or excess fat control of external body parts or body organs on a selective basis (such as “double chin”, arms, hips, “love handles”, thigh, cheeks, eye zone, neck, and abdomen), especially in combination with additional cosmetic benefits, such as antioxidant protection, sunscreen, antiaging, skin wrinkle reduction, deep moisturization, and such is currently not known. The discovery of such is expected to open a vast new consumer market for such combination compositions. The object of an invention to develop such technologies of combination products suitable for topical applications for external body organs or body parts on a selective basis would thus be highly desirable.

### SUMMARY OF INVENTION

I have discovered cosmetic or topical pharmaceutical compositions for external body part or organ slimming, firming, cellulite reduction, fat-reduction, and obesity control benefits that are in synergistic combination with benefits for the treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, spider veins reduction, blood microcirculation (venous insufficiency) improvement, UVA/UVB protection of skin, and skin redness reduction. These compositions thus provide multiple combinations of skin and external body part or organ enhancement benefits that can be selective and specific for external body parts and organs such as face, chin, cheeks, arms, “love handles” in abdomen area, eye lids and eye zone, neck, breasts, thighs, and hips. These compositions include a body beneficial composition selected from certain nutraceutical, cosmetic, and pharmaceutical ingredients, a composition to promote collagen and elastin synthesis in the skin, and a cosmetically or pharmaceutically acceptable delivery system.

This invention thus relates to combinations of certain nutraceutical ingredients with certain cosmetic or active drug compositions to provide external body part or organ selective, specific and synergistic topical benefits. The nutraceutical ingredients that provide external body part or organ slimming, body part firming, body part cellulite reduction, body part fat-reduction, and body part obesity control benefits are thus combined with cosmetic or drug ingredients that provide synergistic treatment and regulation of topical disorders of skin, such as skin aging, wrinkles, acne, rosacea, age-spots, striae distensae (stretch marks), pimples, skin infections and lesions, varicose veins, venous insufficiency, and skin redness.

In a further respect, this invention relates to topical compositions that include certain nutraceutical ingredients in combination with certain cosmetic antioxidant ingredients to provide external body part slimming, firming, cellulite reduction, or fat reduction benefits synergistic with antiaging and wrinkle-reduction benefits.

In a further respect, this invention relates to topical compositions that include certain nutraceutical ingredients in combination with certain cosmetic hydroxy acids (alpha hydroxy acids, beta-hydroxy acids, and poly hydroxy acids) to provide external body part slimming, firming, cellulite reduction, or fat reduction benefits synergistic with skin surface rejuvenating, skin exfoliating, skin surface restructuring, antiaging, and wrinkle-reduction benefits.

In a further respect, this invention relates to topical compositions that include certain nutraceutical ingredients in combination with certain cosmetic skin whitening ingredients to provide external body part slimming, firming, cellulite reduction, or fat reduction benefits synergistic with skin surface whitening, skin surface brightening, age-spot reduction, and antiaging benefits.

In a further respect, this invention relates to topical compositions that include certain nutraceutical ingredients in combination with certain vitamins to provide external body part slimming, firming, cellulite reduction, or fat reduction benefits synergistic with skin surface whitening, skin surface brightening, age-spot reduction, anti-wrinkle, antiaging, pimple reduction, rosacea control, and acne control benefits.

In a further respect, this invention relates to topical compositions that include certain nutraceutical ingredients in combination with certain hormones to provide external body part slimming, firming, cellulite reduction, or fat reduction benefits synergistic with anti-wrinkle, antiaging, skin surface restructuring, skin surface rejuvenating, pimple reduction, rosacea control, stretch marks reduction, and acne control benefits.

In a further respect, this invention relates to topical compositions that include certain nutraceutical ingredients in combination with certain sun screen and sun block ingredients to provide external body part slimming, firming, cellulite reduction, or fat reduction benefits synergistic with UVA/UVB protection, anti-wrinkle, antiaging, skin surface restructuring, and skin surface rejuvenating benefits.

In a further respect, this invention relates to topical compositions that include certain nutraceutical ingredients in combination with certain antibacterial and antifungal ingredients to provide external body part slimming, firming, cellulite reduction, or fat reduction benefits synergistic with acne control, skin protective, wound healing, rosacea control, and stretch marks reduction benefits.

In a further respect, this invention relates to topical compositions that include certain nutraceutical ingredients in combination with certain vasodilating and vasoconstricting ingredients to provide external body part slimming, firming, cellulite reduction, or fat reduction benefits synergistic with vascular insufficiency improvement, blood microcirculation (venous insufficiency) improvement, spider veins control, acne control, and rosacea control benefits.

### DETAILED DESCRIPTION

Most weight loss dietary compositions, pharmaceuticals, and nutraceutical aids are designed to decrease the amount of body fat in an individual by decreasing the individual's appetite for food, decreasing the amount of food absorption in the individual, slowing down the rate of fatty acid synthesis within the body, or increasing the rate of catabolism of fatty acids. The control of body fat, and various biochemical mechanisms that can be utilized for the management of body fat, control of fatty tissue, maintenance of lean body mass, and slimming of body parts and body organs has been described in detail in U.S. patent application Ser. No. 20,010,041,708 (to Yuan-Di Chang Halvorsen, et al.), U.S. Pat. No. 5,804,596 (to Majeed et al.), U.S. patent application Ser. No. 20,020,187,943 (to Majeed et al.), and U.S. patent application Ser. No. 20,020,112,253 (to S J. Wakil et al.). The following are some examples of weight loss products and their mechanisms. Dexfenfluramine increases the brain levels of serotonin, a neurotransmitter and neurohormone that quell the appetite. Sibutramine also increases the levels of serotonin, as well as noradrenaline, and works to quell the appetite. Neuropeptide Y inhibitors curb the appetite, as well as stimulating the body to burn more sugars and less fat. Bromeriptine mimics the neurotransmitter dopamine, and may reduce blood sugar and fat production by the liver. Leptin, a hormone generated by adipocytes, affects the hypothalamus. Cholecystokinin, a hormone and neurotransmitter, acts to reduce appetite. Butabindide blocks an enzyme that inactivates cholecystokinin. Orlistat interferes with pancreatic lipase, which results in poor absorption of dietary fat. Insulinotropin is a glucagon-like hormone which prevents obesity by slowing down the emptying of the stomach. Bta-243 stimulates beta-adrenergic receptors on adipocytes, with a resulting increase in the burning of fatty acids. Troglitazone is a synthetic hormone which signals muscle cells to utilize fat for energy, rather than sugars. Cytokine regulators change the activity of hormone-like cytokines and alter the communication among cells, resulting in weight loss. Hydroxycitric acid acts as an inhibitor of enzyme citrate lyase, which subsequently slows down the synthesis of fatty acids and increases the rate at which fatty acids are burned.

The problem with most dietary and nutraceutical compositions is that they are not selective for a body part or organ. Objectionable taste, incomplete absorption from the digestive system, degradation of the active principles present in such compositions by digestive enzymes, and metabolic alteration of those active principles during absorption into the bloodstream and transport to fatty organ are additional problems with such orally administered compositions. These problems are multiplied and magnified when combinations of compositions to achieve complementary therapies or benefits are desired, such as slimming or fat reduction of with antiaging and vascular improvement benefits, slimming of “double chin” with age-spot reduction of face and neck, slimming of thighs and legs with varicose veins reduction, and other such combination benefits. Since orally administered therapies or compositions generally do not provide benefits selective to certain targeted external body parts or organs, the development of topical compositions to achieve such combination benefits for selective application to external body parts or organs is highly desirable. The present invention discloses compositions to achieve such external body part or organ selective combination treatment compositions that provide synergistically enhanced benefits for all combinations of such desirable benefits.

The present invention provides for external body part or organ slimming, body part firming, body part cellulite reduction, body part fat-reduction, and body part obesity control benefits synergistically combined with treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, varicose and spider veins reduction, blood microcirculation improvement, UVA/UVB protection of skin, and skin redness reduction. It is achieved by combining the following three components;(i)At least one body beneficial composition, selected from certain nutraceutical ingredients, that provides external body part or organ slimming, firming, cellulite reduction, fat-reduction, and obesity control benefits,(ii)At least one cosmetic or drug composition that provides treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, varicose and spider veins reduction, blood microcirculation improvement, UVA/UVB protection of skin, and skin redness reduction, (iii)At least one composition that promotes collagen and elastin synthesis in the skin, and (iv)A cosmetically or pharmaceutically acceptable delivery system or carrier base.

It is surprisingly found in the present invention that all the benefits that are obtained by the combination of nutraceutical composition mentioned in section (i) above and cosmetic or drug composition mentioned in section (ii) above are synergistically enhanced by the inclusion of a collagen and elastin promoting composition mentioned in section (iii) above. Moreover, these compositions are not limited by any specific pH or viscosity conditions of cosmetically or pharmaceutically acceptable delivery system or carrier base mentioned in section (iv) above.

The nutraceutical compositions that provide external body part or organ slimming, body part firming, body part cellulite reduction, body part fat-reduction, and body part obesity control benefits include Forskohlin extract (from Coleus forskohlin plant), Hydroxycitric acid, (from Garcinia cambogia, and plants of Garcinia family), L-Carnitine, Creatine, Human growth hormone (HGH), Chromium picolinate, Kola seed extract, Caffeine, Niacinamide, Psyllium husk, Chitosan, Lipoprotein complexes, Polyphenols, Gymnemic acid, Pyruvic acid and Pyruvate salts, salts of Hydroxycitric acid, Phaseolamin (from Phaseolus vulgaris extract), DHEA, Chitosan, Theophylline, Theobromine (or salts thereof such as Aminophylline), Roselle tea extract, Arabinose, Inosine, Adenosine, Fructose-1,6-diphosphate, Adenosine triphosphate (ATP), Adenosine diphosphate (ADP), NDGA, Indomethacin, ETYA, SHA, Baicalein, 3,4-Dihydroxycinnamic acid, Cinnamyl-3,4-dihydroxy-a-cyanocinnamate, Gossypol, 5,6-Dehydro arachidonic acid, Baeomycesic acid, Baicalein, 3,4-Dihydroxyphenyl ethanol, 4,5-Dehydro docosahexaenoic acid, Eicosatriynoic acid, 5-HETE-lactone, 9,12-Octadecadiynoic acid, alpha-Pentyl-3-(2-quinolinylmethoxy)-benzenemethanol, 3-Amino-1-(m-(trifluoromethyl)phenyl)-2-pyrazoline, 6,9-Diepoxy-6,9-phenylimino-delta 6,8-prostaglandin I, Extract of the plant of genus Tephrosia, Natriuretic peptide, Laminaria extract, Extract from berries of Panax genus plant, Gymnema sylvestre extract, 9-cis, 11-trans Conjugated linoleic acid and 10-trans, 12-cis conjugated linoleic acid isomers (conjugated linoleic acid, CLA), Calcium salts, Synephrine, Hordenine, Octopamine, Tyramine, n-Methyltyramine, Val-Tyr-Pro or Val-Thr-Leu peptide, Azaftig, inhibitors of acc2 acetyl-COA carboxylase enzyme, Extract of Climbing ivy (Hedera helix), Extract of Arnica (Arnica montana), Extract of Rosemary (Rosmarinus officinalis), Extract of Marigold (Calendula officinalis), Extract of Sage (Salvia officinalis), Extract of Ginseng (Panax ginseng), Extract of St. Johns-wart (Hypericum perforatum), Extract of ruscus (Ruscus aculeatus), Extract of meadowsweet (Filipendula ulmaria), and Extract of Orthosiphon (Ortosifon stamincus). These ingredients can be either used alone or in combination with each other. The quantities of these ingredients are not limited to any specific number or range of numbers. It is recommended that compositions at several varying levels of ingredient or ingredient combinations be made and tested for their specific applications and benefits desired.

The cosmetic or drug compositions that provide treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, varicose and spider veins reduction, blood microcirculation improvement, UVA/UVB protection of skin, and skin redness reduction can be selected from a great number of ingredients and compositions, some of which are already well known in the prior art. However, many of such ingredients or compositions known in the prior art have not been disclosed in any combination, and especially in any synergistically enhanced compositions that provide superior combination benefits.

Presently, there are millions of people around the world who suffer from mild-to-moderate chronic venous insufficiency (varicose veins) of the legs. This common condition is characterized by an inadequacy of the venous circulation to return blood from the legs to the heart. The lack of adequate venous return results in venous stasis and an increased pressure within the venous circulation, promoting the development of edema and water retention. Chronic venous insufficiency is a functional disorder caused by persistent inadequacy of the venous return and is characterized clinically by edema, skin changes and subjective complaints such as tired, heavy legs, pain or tingling sensations, which are typically amplified by excess body weight, standing upright, and by high ambient temperatures. For people with weight problem, especially those with fatty legs, the spider veins and poor circulation problems are even more magnified. It would thus be beneficial to provide a cure for varicose and spider veins and poor microcirculation in external body parts such as legs and arms in combination with body shape management (localized fat reduction and slimming of external body parts). Several blood microcirculation improvement agents are already known that includes Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), and Amni visnaga extract. Recently, U.S. Pat. No. 6,485,727 (to Esperester et al.) has disclosed extract of Red Vine (Vitis-Vinifera) leaves for venous insufficiency by oral administration. The topical benefits of this ingredient for the cure of spider veins and venous insufficiency are now claimed in the present invention. It has been surprisingly discovered in the present invention that the efficacy of a combination of blood microcirculation improvement ingredients and body fat reduction (body slimming) ingredients is synergistically enhanced by the inclusion of a composition to promote collagen and elastin synthesis in the skin.

The reduction of the signs of skin aging, reduction of skin wrinkles, and reduction of age-spots occupies greater emphasis for the mankind now than ever before in human history. The incorporation of botanical antioxidants in cosmetic products is gaining popularity due to anti aging, wrinkle reduction, and other skin tone enhancement benefits, concordant to their use as nutritional supplements. Cosmetic products formulated with familiar antioxidants (vitamin E, Coenzyme Q10, ascorbic acid, alpha-lipoic acid, and soy isoflavones, etc.) have appeared in the marketplace. The design of a topical antioxidant product offers challenges: A wide spectrum antioxidant product should control cellular oxidation resulting from biochemical mechanisms including oxygen, free radicals, UV, atmospheric pollutants, oxidative enzymes, catabolic oxidation, and chemical oxidation. A combination of antioxidants is more effective than a single antioxidant on an equal weight basis due to antioxidant cascade mechanism. The combination of antioxidant ingredients should be selected from different chemical classes to control cellular oxidation resulting from various biochemical mechanisms. A number of compositions or ingredients suitable to provide such benefits can be selected from Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. It has been surprisingly discovered in the present invention that the efficacy of a combination of compositions or ingredients for the reduction of the signs of skin aging, reduction of skin wrinkles, and reduction of age-spots and body fat reduction (body slimming) ingredients is synergistically enhanced by the inclusion of a composition that promotes collagen and elastin synthesis in the skin.

The skin whitening, skin brightening, and age-spot removal is another greatly desirable benefit for cosmetic compositions. Skin whitening, skin brightening, or age-spot reducing composition is selected from hydroquinone, arbutin, hydroquinone derivatives, Paper Mulberry extract (Broussonetia kazinoke), Mitracarpe extract (Mitracarpus scaber), Bearberry extract (Arctostaphylos uva ursi), Yellow Dock extract (Rumex crispus and Rumex occidentalis), Glutathione, Leucocyte extract, Aspergillus orizae extract (Aspergillus orizae), Licorice Root extract (Glycyrrhiza glabra), Rosmarinic acid (Rosmarinus officinalis), Tetrahydrocurcumin, Green Tea extract (Camellia sinensis), Yohimbe extract (Pausinystalia yohimbe), Ecklonia cava extract, niacinamide, Hydroxytetronic acid, Spondias mombin extract, Maprounea guianensis extract, Walteria indica extract, Gouania blanchetiana extract, Cordia schomburgkii extract, Randia armata extract, and Hibiscus furcellatus extract.

Additional cosmetically or pharmaceutically beneficial compositions that can be combined with external body part or organ slimming, firming, cellulite reducing, fat-reducing, and obesity control benefits includes, but not limited to, UV protection, acne control, rosacea control, antimicrobial and antifungal, and other such benefits. These are further covered in the Claims section of the present invention.

It is a well known problem that the weight loss, slimming, or fat reduction usually causes skin wrinkles, as the skin takes much longer to adjust to the shrinkage of tissue from such fat reduction actions. A combination of weight loss, slimming, or other such fat reduction compositions with antiaging and wrinkle reduction benefits is thus very difficult, since such weight or fat reduction compositions may also cause skin wrinkle formation. It has been surprisingly discovered in the present invention that the efficacy of a combination of compositions or ingredients for the skin whitening, skin brightening, and reduction of age-spots and body fat reduction (body slimming) ingredients is synergistically enhanced by the inclusion of a composition that promotes the formation of collagen and elastin in the skin. Since many of such collagen and elastin boosting compositions also possess antioxidant and anti-inflammatory affects, it has also been found that such compositions also synergistically enhance other antiaging and wrinkle reduction compositions present in combination with weight loss, slimming, or fat reduction compositions. As is also evident from these findings, many such compositions that have antioxidant or anti-inflammatory affects may provide a dual benefit in the compositions of the present invention. Although not bound by any theory, it appears that the initial skin wrinkles that are first formed by the affects of body part slimming or fat reduction compositions are visibly reduced by enhanced collagen and elastin formation in the skin. This permits skin to then naturally adjust itself by shedding excess protein and other tissue thus reducing such initially formed wrinkles, at which point external body part or organ appears slim and toned.

The cosmetically or pharmaceutically acceptable delivery system or carrier base compositions can be in any convenient form, such as a clear liquid, serum, lotion, cream, gel, mask, spray, cleanser, shampoo, shower gel, and conditioner, etc. Such compositions can be pre-made, and other ingredients disclosed in the present invention can be added as a solution in an appropriate solvent, or added directly into such pre-made carrier base compositions. Additionally, such delivery system or carrier base compositions can also be made in situ, as illustrated in several examples provided in this invention. Moreover, the ingredients of the present invention can also be added to already formulated products. For example, in a skin lotion that has already been made, the ingredients for cellulite reduction, antiaging benefits, and collagen and elastase promoters can all be added in the amounts specified in the present invention to provide added benefits from such lotion product. As can be seen from this description, the present invention also provides several convenient methods for manufacturing the compositions of present invention.

It is to be further explained that the quantities of any ingredients and compositions of the present invention are not limited to any specific amounts. The quantities shall be based on safe and effective amounts of such ingredients and compositions, since the ingredients and compositions of the present invention represent a very large class of materials that are cosmetic, nutraceutical, or pharmaceutical groups that have a very widely distributed safety and efficacy range for their various efficacies. It is thus nearly impossible to establish any specific limits on the quantities of such ingredients, especially when such ingredients are used in compositions of synergistic combinations. It is to be noted from various examples of the present invention that it is easily possible to include various compositions for combination benefits in amounts from 0.0001% to in excess of 40%. This is because of current popular consumer trend for such amounts. It is to be further noted that such amounts are not thus assumed to be limited to about 40%, since in future the consumer trend may require higher amounts of such compositions. In future, such amounts may depend on several factors that include safety, efficacy, bioavailability, cost, performance, consumer appeal, competition, stability, and such. Since such conditions are not within the control of present inventor, it is preferred that any amounts of such beneficial combination compositions are not artificially limited to certain specific number or a range of numbers.

### EXAMPLES

The following examples are presented to illustrate presently preferred practice thereof. As illustrations they are not intended to limit the scope of the invention. All quantities are in weight %.

### Example 1

### Chitosan Facial Mask Composition Including the Ingredients for the Reduction of Wrinkles and Excess Fat on Cheeks and Eyelids

Ingredient % 1. Chitosan52. Lactic Acid53. Glycerin184. Water65.85. Hydroxycitric acid56. Niacinamide0.57. Glutathione0.28. Preservatives0.5Procedure: Mix 1, 2, and 3 to a paste.

Mix 4 to 8 separately to a clear solution. Add this to main batch and mix.

A clear gel product is obtained. It is applied on the face and neck and left for 10 to 30 minutes, then rinsed off.

### Example 2

### Psyllium Facial Mask Including the Ingredients for Antiaging and Double Chin Reduction

Ingredient % 1. Psyllium husk powder102. PEG-6303. Forskohlin14. Calcium Sulfate55. Soybean Fibers56. Oat Protein57. Carnosine0.58. Water439. Preservatives0.5Procedure. Mix 1 to 6 to a paste. Mix separately 7 to 9. Add to main batch and mix to a paste. A thin dough-like product is obtained. It is applied on the face and neck area with fingers. After 10 to 30 minutes, it is washed off.

Example 3

### Chitosan Facial Mask with Ingredients for Skin Whitening and Reduction of Fatty Tissue in Puffy Eye Lids

Ingredient % 1. Chitosan52. Lactic Acid53. PEG-6204. Forskohlin (water soluble) 15. Tetrahydrocurcumin0.16. Licorice Root Extract0.57. Water50.98. Hydroxycitric acid59. Hydroquinone210. Soybean Fibers511. Oat Protein512. Preservatives0.5Procedure. Mix 1 to 6 to a paste. Mix separately 7 to 12. Add to main batch and mix to a paste. It is applied on the face and neck area with fingers. After 10 to 30 minutes, it is washed off.

### Example 4

### Chitosan Serum with Ingredients for Age Spot Removal and Double-Chin Reduction

Ingredient % 1. Cognis Hydagen CMF (Chitosan & water)502. Hydroxycitric acid53. Coleus forskohlii extract, water soluble24. Deionized Water27.55. Glycerin106. Niacinamide Ascorbate57. Preservative0.5Procedure. Mix all ingredients together. A clear serum is obtained, with syrupy appearance.

### Example 5

### Foaming Facial Mask with Ingredients for Deep Cleansing, Rosacea reducing, and Excess Facial Oil Removing Effects.

Ingredient% 1. Chitosan52. Citric acid 53. Glycerin184. Water58.85. Sodium Cocoyl Isethionate 106. Polydimethylsiloxane 27. Emblica (Phyllanthus emblica) extract 0.28. Horse Chestnut extract0.59. Preservatives0.5Procedure: Mix 1, 2, and 3 to a paste.

Mix 4 to 9 separately to a clear solution. Add this to main batch and mix.

A thick, translucent gel product is obtained. It is applied on the face and neck and left for 10 to 30 minutes, then rinsed off. During rinsing, much foam is generated that provides cleansing action.

### Example 8

### The Preparation of an Anti-Aging Night Cream with Wrinkle Reduction, Skin Whitening, Antioxidant Protection, and Cheek Fat Reduction Ingredients

Ingredient % Water 81.15Carbomer0.2GMS-SE 2.0Stearic Acid 3.0Cetyl Alcohol 1.5Glycerin 1.0jojoba Oil 0.1Sweet Almond Oil 0.1Sesame Oil 0.2Apricot Kernel Oil 0.2Panthenol 0.1Glydant Plus (Preservative)0.2Dimethicone2.0Vitamin E Acetate0.1Vitamin A Palmitate 0.1Coleus forskohlii extract5.0Garcinia cambogia extract2.0Fructose-1,6-diphosphate0.5Glutathione0.05Fragrance0.15Botanical Extract0.25Procedure: All ingredients, except fragrance and botanical extract, were mixed and heated at 70 to 80 C, then cooled to room temperature. Fragrance, and botanical blends were all added to the main batch and the batch mixed. An off-white cream was obtained.

### Example 9

### Face & Body Cleanser with Surfactant, Facial Skin Toning, and Skin Regenerating Ingredients.

Ingredient % Water 65.04Germall II (preservative)0.1Kathon CG (preservative) 0.06Sodium Lauryl Sulfate18.0Cocamidopropyl betaine10.0Hydroxy Citric Acid1.5Carnitine2.5Ellagic acid0.5Niacinamide Ascorbate0.5Fructose-1,6-diphosphate0.2Fragrance0.5Botanical Extracts 0.2Procedure: All ingredients were mixed in a tank. A light yellow clear liquid product was obtained.

### Example 10

This example illustrates the preparation of a facial acne treatment cream that contains salicylic acid as a drug active ingredient with the enhancement of its efficacy by the inclusion of niacinamide salicylate and niacinamide lipoate and cellulite reduction ingredients.

Ingredients % 1. Polyethylene glycol (PEG-6) 63.022. Aristoflex AVC (ammonium acryloyldimethyltaurate/vp copolymer) 1.03. Deionized water 15.04. Phaseolamine 2.05. Carnosine 0.56. Creatine 2.07. Killitol (preservative) 0.38. Jeesilc 6056 (polydimethylsiloxane)10.09. Actiplex (plant extracts blend) 0.110. Titanium dioxide 0.211. Niacinamide Salicylate2.612. Niacinamide Lipoate3.28Procedure: Mix 2 and 3 till clear. Add all other ingredients except 9. Heat at 50 to 60 C for 30 minutes till all solids dissolved. Cool to room temperature with slow mixing. Add 9. Mix till homogeneous. A white cream is obtained.

### Example 11

This example illustrates the preparation of a 40.0% serum for a very high potency skin rejuvenating and cellulite reduction applications.

Ingredients % 1. Carbowax (PEG-6)30.02. Deionized Water28.03. Hydroxycitric acid20.04. Coleus forskohlii extract (water soluble) 20.05. Carnosine 1.06. Ascorbic acid1.0Process: Mix all ingredients till a clear solution is obtained.

### Example 12

This example illustrates the preparation of a skin whitening formula that contains hydroquinone as the drug active ingredient, with niacinamide ascorbate and niacinamide lactate also added to boost the skin-whitening efficacy.

Body slimming and cellulite reducing ingredients are also included. Ingredient % 1. Carbowax 300 (PEG-6)62.4 2. Aristoflex AVC 0.8 3. Deionized Water 15.0 4. Chromium picolinate 0.5 5. Creatine 1.5 5. Hydroquinone 4.0 6. Jeechem HPIB (silicone blend)10.07. Killitol (preservative) 0.38. Garcinia cambogia extract 2.09. Niacinamide Ascorbate2.010. Niacinamide lactate1.5Process: Mix 2 and 3 till a clear gel is formed. Add 1, 7, and 8 and heat at 50 to 60 C. Add all other ingredients with mixing. Cool to room temperature. A translucent cream is obtained.

### Example 13

### The Preparation of a High Potency Varicose Veins Reducing Composition with “Love Handle” and Thighs Slimming Ingredients

Ingredients % Escin2.5Esculin2.5Ruscogenins2.5Boswellia serrata extract2.0Methylpropanediol69.0Vitamin K0.5Tetrahydrocurcumin0.5Coleus forskohlii extract10Water10Phenoxyethanol0.5Procedure: All ingredients were mixed with heating at 50 to 60 C. A serum product was obtained which was quick absorbing.

### Example 14

This example shows an antiaging, free-radical fighting facial cleanser composition with ingredients added for facial fat reduction and skin brightening.

Ingredient % Glycerin 38.3Methyl paraben (preservative)0.2Ascorbic Acid9.0Niacinamide (nicotinamide)6.0Deionized Water15.0Phenoxyethanol (preservative) 0.9Tauranol I-78-6 (Sodium Cocoyl Isethionate) (surfactant) 20.0Tauranol ws conc. (Sodium Methyl Cocoyl Taurate) (surfactant)5.0Actiplex 2789 (Extract of various plants)0.1 Garcinia cambogia extract2.5Coleus forskohlii extract2.5Fragrance 0.5Procedure: Mix deionized water, ascorbic acid, and niacinamide in a tank separately. A clear solution is obtained. All of the other ingredients are then added, except fragrance, and the mixture is heated and stirred at 60 to 70 degrees C. for about five to ten minutes until the mixture is homogenous. The homogeneous mixture is cooled to room temperature, and fragrance is added with mixing. A paste-like product is formed.

Example 15

This example illustrates a synergistic composition with anti-acne, skin whitening, anti-wrinkle, and cellulite reduction ingredients.

Ingredients % 1. PEG-645.52. Aristoflex AVC 1.03. Glycerin 5.04. Deionized Water 20.05. Vitamin EAcetate2.06. Geogard 221 (Preservative)0.57. Dimethicone 4.012. Dimethiconol4.013. Cetyl Dimethicone Copolyol2.014. Benzyl Niacin Ascorbate3.015. Benzyl Niacin Lactate3.016. Benzyl Niacin Mandelate3.017. Phaseolamin2.018. Coleus forskohlii extract5.0Process: Mix 2, 3, and 4 till a clear gel is formed. Add all other ingredients and mix. Heat at 40 to 50 C for 30 minutes. Cool to room temperature. A translucent cream is obtained. It is rapidly absorbed upon application to skin.

### Example 17

### SPF 25 Sunscreen Lotion with High performance Cellulite Reduction Ingredients for Abdomen, Thighs, and Legs

Ingredient % 1. Deionized Water60.52. Glycerin5.03. GMS-SE3.04. Stearic Acid3.05. Arlacel 1655.06. Cetyl Alcohol0.57. Sweet Almond Oil0.58. Titanium Dioxide2.09. Ethylhexyl Methoxycinnamate 7.510. Coleus forskohlii extract10.011. Kaempferia galanga extract2.512. Preservative 0.5Procedure. Mix 1 to 8 and heat at 80 to 90 C till all solids have melted. Cool to 50 to 60 C and add 9 to 11. Cool with mixing to 35 to 40 C. Add 12. Mix. Adjust pH. Homogenize, and then cool to room temperature. A white lotion is obtained.

I claim:

1. A synergistic cosmetic or pharmaceutical composition for external body part or organ slimming, firming, cellulite reduction, fat-reduction, and obesity control benefits synergistically combined with treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, varicose and spider veins reduction, blood microcirculation improvement, UVA/UVB protection of skin, and skin redness reduction comprising:
(i) at least one body beneficial composition, selected from certain nutraceutical ingredients, that provides external body part or organ slimming, body part firming, body part cellulite reduction, body part fat-reduction, and body part obesity control benefits,
(ii) at least one cosmetic or drug composition that provides treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, varicose and spider veins reduction, blood microcirculation improvement, UVA/UVB protection of skin, and skin redness reduction,
(iii) at least one composition to promote collagen and elastin synthesis in the skin, and
(iv) a cosmetically or pharmaceutically acceptable delivery system or carrier base composition.

2. A composition according to claim 1 wherein the body beneficial nutraceutical ingredient that provides external body part or organ slimming, body part firming, body part cellulite reduction, body part fat-reduction, and body part obesity control benefits is selected from the group consisting of Forskohlin extract (from Coleus forskolii plant), Hydroxycitric acid, (from Garcinia cambogia, and plants of Garcinia family), L-Carnitine, Creatine, Human growth hormone (HGH), Chromium picolinate, Kola seed extract, Caffeine, Niacinamide, Psyllium husk, Chitosan, Lipoprotein complexes, Polyphenols, Gymnemic acid, Pyruvic acid and Pyruvate salts, salts of Hydroxycitric acid, Phaseolamin (from Phaseolus vulgaris extract), DHEA, Chitosan, Theophylline, Theobromine (or salts thereof such as Aminophylline), Roselle tea extract, Arabinose, Inosine, Adenosine, Fructose-1,6-diphosphate, Adenosine triphosphate (ATP), Adenosine diphosphate (ADP), NDGA, Indomethacin, ETYA, SHA, Baicalein, 3,4-Dihydroxycinnamic acid, Cinnamyl-3,4-dihydroxy-a-cyanocinnamate, Gossypol, 5,6-Dehydro arachidonic acid, Baeomycesic acid, Baicalein, 3,4-Dihydroxyphenyl ethanol, 4,5-Dehydro docosahexaenoic acid, Eicosatriynoic acid, 5-HETE-lactone, 9,12-Octadecadiynoic acid, alpha-Pentyl-3-(2-quinolinylmethoxy)-benzenemethanol, 3-Amino-1-(m-(trifluoromethyl) phenyl)-2-pyrazoline, 6,9-Diepoxy-6,9-phenylimino-delta 6,8-prostaglandin I, Extract of the plant of genus Tephrosia, Natriuretic peptide, Laminaria extract, Extract from berries of Panax genus plant, Gymnema sylvestre extract, 9-cis, 11-trans Conjugated linoleic acid and 10-trans, 12-cis conjugated linoleic acid isomers (conjugated linoleic acid, CLA), Calcium salts, Synephrine, Hordenine, Octopamine, Tyramine, n-Methyltyramine, Val-Tyr-Pro or Val-Thr-Leu peptide, Azaftig, inhibitors of acc2 acetyl-COA carboxylase enzyme, Extract of Climbing ivy (Hedera helix), Extract of Arnica (Arnica montana), Extract of Rosemary (Rosmarinus officinalis ), Extract of Marigold (Calendula officinalis), Extract of Sage (Salvia officinalis), Extract of Ginseng (Panax ginseng), Extract of St. Johns-wart (Hypericum perforatum), Extract of ruscus (Ruscus aculeatus), Extract of meadowsweet (Filipendula ulmaria), Extract of Orthosiphon (Ortosifon stamincus), and combinations thereof. The quantities of such compositions can be safe and effective amounts as needed, and not limited to any specific limits.

3. A composition according to claim 1 wherein the composition to promote collagen and elastin in the skin is selected from Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), and combinations thereof. The quantities of such compositions can be safe and effective amounts as needed, and not limited to any specific limits.

4. A composition according to claim 1 wherein the cosmetic or drug ingredient that provides treatment of skin aging, skin wrinkles reduction, skin exfoliating, treatment of acne, treatment of rosacea, age-spots reduction, skin surface whitening, skin surface brightening striae distensae (stretch marks) reduction, treatment of pimples, treatment of skin infections and lesions, spider veins reduction, blood microcirculation improvement, UVA/UVB protection of skin, and skin redness reduction is selected from a group consisting of antioxidants, various hydroxy acids (alpha hydroxy acids, beta hydroxy acids, and polyhydroxy acids), vitamins, hormones, skin whitening agents, UVA/UVB sunscreens, antimicrobial agents, antifungal agents, blood microcirculation improvement agents (vasodilatory or vasoconstrictive), skin protectant drug actives, and combinations thereof. The quantities of such compositions can be safe and effective amounts as needed, and not limited to any specific limits.

5. A composition according to claim 1 wherein a cosmetically acceptable delivery system can be in the form of a lotion, cream, gel, spray, thin liquid, body splash, mask, serum, solid cosmetic stick, lip balm, shampoo, liquid soap, bar soap, bath oil, cologne, hair conditioner, salve, collodion, impregnated patch, impregnated strip, skin surface implant, and any other such cosmetically or pharmaceutically acceptable topical delivery forms.

6. A composition according to claim 1 wherein a cosmetically acceptable delivery system can be prepared separately and all other ingredients according to claim 1 can be added separately in the form of mixtures, solutions, suspensions, or emulsions to facilitate the manufacturing of such compositions.

7. A composition according to claim 4 wherein the skin beneficial hydroxy acid is selected from the group consisting of salicylic acid, lactic acid, glycolic acid, malic acid, mandelic acid, ascorbic acid, ascorbyl phosphoric acid, hydroxycitric acid, hydroxytetronic acid, citric acid, aleuritic acid, ellagic acid, rosmarinic acid, chlorogenic acid, polysulfonic acid, and hyaluronic acid (HYA). The quantities of such compositions can be safe and effective amounts as needed, and not limited to any specific limits.

8. A composition according to claim 4 wherein the skin beneficial skin whitening agent is selected from hydroquinone, arbutin, hydroquinone derivatives, Paper Mulberry extract (Broussonetia kazinoke), Mitracarpe extract (Mitracarpus scaber), Bearberry extract (Arctostaphylos uva ursi), Yellow Dock extract (Rumex crispus and Rumex occidentalis), Glutathione, Leucocyte extract, Aspergillus orizae extract (Aspergillus orizae), Licorice Root extract (Glycyrrhiza glabra), Rosmarinic acid (Rosmarinus officinalis), Tetrahydrocurcumin, Green Tea extract (Camellia sinensis), Yohimbe extract (Pausinystalia yohimbe), Ecklonia cava extract, niacinamide, Hydroxytetronic acid, Spondias mombin extract, Maprounea guianensis extract, Walteria indica extract, Gouania blanchetiana extract, Cordia schomburgkii extract, Randia armata extract, Hibiscus furcellatus extract, and combinations thereof. The quantities of such compositions can be safe and effective amounts as needed, and not limited to any specific limits.

9. A composition according to claim 4 wherein the skin beneficial antioxidant composition is selected from Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, Emblica extract, and combinations thereof. The quantities of such compositions can be safe and effective amounts as needed, and not limited to any specific limits.

10. A composition according to claim 4 wherein the UVA/UVB sunscreen composition is selected from Titanium dioxide, Zinc oxide, Galanga extract (Kaempferia galanga), Benzophenone-3, Benzophenone-4, Ethylhexyl Methoxycinnamate, Homosalate, Ethylhexyl salicylate, Octocrylene, Menthyl anthranilate, Avobenzone, Lawsone, Sulisobenzone, Trolamine salicylate, Lawsone, Glyceryl aminobenzoate, Cinoxate, and PABA. and combinations thereof. The quantities of such compositions can be safe and effective amounts as needed, and not limited to any specific limits.

11. A Composition According to claim 4 wherein the blood microcirculation improvement composition is selected from Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof. The quantities of such compositions can be safe and effective amounts as needed, and not limited to any specific limits.

12. A composition according to claim 4 wherein the antimicrobial composition is selected from Berberine, Triclosan, Triclocarban, various Tritons (quaternary ammonium compounds), Benzyl Alcohol, Dehydroacetic Acid, Phenoxyethanol, and combinations thereof. The quantities of such compositions can be safe and effective amounts as needed, and not limited to any specific limits.

13. A composition according to claim 4 wherein the vitamin composition is selected from Vitamin A, members of Vitamins B group, Vitamin C, Vitamin D, Vitamin E, Vitamin K, Carotenes, Biotin, Folic Acid, and combinations thereof. The quantities of such ingredients can be safe and effective amounts as needed, and not limited to any specific limits.

14. A composition according to claim 4 wherein the hormone composition is selected from progesterone, androsterone, dehydroepiandrosterone (DHEA), Pregnenolone, androstenedione, melatonin, testosterone, and combinations thereof. The quantities of such compositions can be safe and effective amounts as needed, and not limited to any specific limits.

15. A composition according to claim 4 wherein the skin protectant drug composition is selected from Allantoin, petrolatum, glycerin, dimethicone, urea, calamine, cocoa butter, kaolin, zinc acetate, zinc carbonate, and combinations thereof. The quantities of such compositions can be safe and effective amounts as needed, and not limited to any specific limits.

16. The compositions according to claim 5 wherein the cosmetically or pharmaceutically acceptable delivery system can be traditional water and oil emulsions, suspensions, colloids, microemulsions, clear solutions, suspensions of nanoparticles, emulsions of nanoparticles, or anhydrous compositions.

17. A composition according to claim 1 wherein additional skin beneficial ingredients, such as skin cleansers, surfactants (cationic, anionic, non-ionic, amphoteric, and zwitterionic), skin and hair conditioning agents, vitamins, hormones, minerals, plant extracts, anti-inflammatory agents, concentrates of plant extracts, emollients, moisturizers, skin protectants, humectants, silicones, skin soothing ingredients, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and like can be added to the formulation. The quantities of such ingredients can be safe and effective amounts as needed, and not limited to any specific limits.
